(19) United States
(12) Patent Application Publication
(10) Pub. No.: US 2007/0161074 A1
(43) Pub. Date: Jul. 12, 2007

(54) DIAGNOSTIC METHOD OF MUCOPOLYSACCHARIDOSES

(75) Inventors: Shunji Tomatsu, St. Louis, MO (US); Toshihiro Oguma, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignees: DAIICHI PHARMACEUTICAL CO., LTD., Tokyo (JP); St. Louis University, St. Louis, MO (US)

(21) Appl. No.: 11/616,586

(22) Filed: Dec. 27, 2006

Related U.S. Application Data
(60) Provisional application No. 60/753,413, filed on Dec. 27, 2005.

(30) Foreign Application Priority Data
Sep. 21, 2006 (JP) 2006-255869

(51) Int. Cl. C12Q 1/34

(52) U.S. Cl. 435/18

(57) ABSTRACT

Provision of a method for accurate diagnosis of mucopolysaccharidoses, including determining the level of glycosaminoglycan in a biological sample with high sensitivity and with ease. A diagnostic method of mucopolysaccharidoses including the following steps (1) and (2): (1) a step including (a) filtering a biological sample with an ultrafiltration filter, digesting the sample on the filter with a glycosaminoglycan-specific enzyme, centrifuging the digested sample to obtain a filtrate, or (b) digesting a biological sample with a glycosaminoglycan-specific enzym, filtering the sample with an ultrafiltration filter to obtain a filtrate, applying the filtrate obtained by (a) or (b) to a liquid chromatograph/mass spectrometer, and analyzing glycosaminoglycan-derived disaccharides, and (2) a step of diagnosing a subject as having mucopolysaccharidosis, chemically diagnosing effect of treatment of mucopolysaccharidoses, or determining types of mucopolysaccharidoses, on the basis of quantitative concentration data and disaccharide composition obtained in step (1).

### FIELD OF THE INVENTION

The present invention relates to a diagnostic method of mucopolysaccharidoses.

### BACKGROUND ART

Mucopolysaccharidoses are a group of lysosomal storage diseases caused by deficiency of the lysosomal enzymes needed to degrade glycosaminoglycans (GAGs). In patients suffering mucopolysaccharidosis, degradation products of mucopolysaccharides systemically accumulate, gradually impairing the functions of tissue and organs. Mucopolysaccharidoses are primarily classified into 7 types depending on the identity of the lacking enzyme. Most mucopolysaccharidosis cases are progressive and accompanied by mental retardation, and in some types of the disease, the clinical outcome is often death in early adult life. Clinical abnormalities primarily include significantly deformed bones, a short neck, joint stiffness and coarse facial features. In addition, diffuse cornea opacification, hearing disorder, liver enlargement, heart diseases, and abnormally low height are observed.

In diagnosis of mucopolysaccharidoses, glycosaminoglycans (hereinafter reffered to as GAG) content of a biological sample, such as blood, is determined. Conventionally known assays of GAGs include the following methods.

JP-A-4-135496 discloses a method of analyzing GAG, which method includes transforming GAG into disaccharides by use of an enzyme that specifically degrades GAG, and analyzing the composition of the resultant disaccharides by means of high performance liquid chromatography (hereinafter referred to as HPLC). Chem. Pharm. Bull. 46 (1), 97 to 101 (1998) discloses a method of analyzing KS, which method includes transforming keratan sulfate (hereinafter referred to as KS) in urine into disaccharides by use of keratanase, which is an enzyme that specifically degrades KS, and analyzing the resultant disaccharides by means of HPLC. Journal of Chromatography B, 765, 151 to 160 (2001) discloses an analysis method of GAG, including hydrolysis of plasma GAG or serum GAG, and formed galactose and aminosugar are analyzed by means of HPLC. Analytical Biochemistry 302, 169 to 174 (2002) discloses an analysis method of chondroitin sulfate (hereinafter referred to as CS), which method include filtration of plasma CS or urine CS through an ultrafiltration filter, followed by degradation of CS into disaccharides with chondroitinase ABC on the filter, and analyzing the disaccharides contained in the filtrate by means of HPLC. Analytical Biochemistry 290, 68 to 73 (2001) discloses a method of analyzing the composition of KS-derived disaccharides, which method includes pretreatment of tissue KS through ethanol precipitation, degrading the pretreated product with keratanase II into disaccharides, followed by liquid chromatography/tandem mass spectrometry of the resultant disaccharides (hereinafter referred to as LC/MS/MS), whereby the KS-derived disaccharide composition is investigated. Journal of Chromatography B, 754, 153 to 159 (2001) discloses an analysis method of the heparan sulfate (HS) derived disaccharide composition, which method includes pretreatment of tissue through ethanol precipitation, degradation into disaccharides by use of an enzyme specifically directed to HS, and injecting the disaccharides by means of LC/MS/MS. JP-A-2003-265196 and Clinica Chimica Acta, 264, 245 to 250 (1997) respectively describe a method of diagnosing mucopolysaccharidoses through measurement of urine GAG using 1,9-dimethylmethylene blue.

Also, JP-A-10-153600 discloses an assay method using a polypeptide that is capable of specifically binding to KS and Hyaluronic acid (hereinafter referred to as HA)-containg molecule.

### DISCLOSURE OF THE INVENTION

### Problem to be Solved by the Invention

However, conventional methods have various problems, including a scatter of measured concentrations, low measurement sensitivity, and intricate pretreatment procedure. Moreover, only one type of GAG can be measured in a single test. Thus, no conventional diagnostic method has been satisfactory for the diagnosis of mucopolysaccharidoses.

Accordingly, the present invention provides a method for accurate diagnosis of mucopolysaccharidoses, including determining the level of glycosaminoglycan in a biological sample with high sensitivity and with ease.

### Means to Solve the Problem

The present inventors have carried out extensive studies with an aim to develop a method for simultaneous measurement of a plurality of glycosaminoglycans in a biological sample with high sensitivity, and have found that accurate diagnosis of mucopolysaccharidoses can be rendered from highly sensitive simultaneous quantification of a plurality of glycosaminoglycans contained in a biological sample, which is realized when use of an ultrafiltration filter and enzymatic digestion performed on the filter is further combined with LC/MS/MS. The present invention has been accomplished on the basis of this finding.

The present invention provides (A) to (E) below.

(A) A diagnostic method of mucopolysaccharidoses including the following steps (1) and (2):
(1) a step including (a) filtering a biological sample with an ultrafiltration filter, digesting the biological sample on the filter with a GAG-specific enzyme, and centrifuging the digested sample to obtain a filtrate, or (b) digesting a sample with with a GAG-specific enzyme, filtering the digested sample with an ultrafiltration filter to obtain a filtrate, applying the filtrate obtained by (a) or (b) to LC/MS/MS, and analyzing GAG-derived disaccharides, and
(2) a step of diagnosing a subject as having mucopolysaccharidosis or determining types of mucopolysaccharidoses, on the basis of quantitative concentration data and disaccharide composition obtained in step (1).

(B) A method as described in (A), wherein, in step (1), the HPLC is performed under such conditions that the analytical column is a carbon graphite column and an alkaline solution is employed as a mobile phase, to thereby elute GAG-derived disaccharides at optimal elution positions that facilitate the MS analysis.

(C) A method as described in (A) or (B), wherein, in step (1), the disaccharides are produced through use of a solution containing, as the GAG-specific degrading enzyme, keratanase II, heparitinase, and chondroitinase B; and KS, HS, and DS are analyzed simultaneously.

(D) A method as described in (A) or (B), wherein, in step (1), the disaccharides are produced using, as the GAG-specific degrading enzyme, any one of keratanase II, heparitinase, and chondroitinase B; and one or two of KS, HS, and DS are analyzed.

(E) A method as described in any one of (A) to (D), wherein, in step (1), the biological sample is selected from among plasma, serum, blood, urine, and body fluid.

### Advantageous Effect of the Invention

Hence, the method of the present invention in its broadest scope provides an accurate, highly sensitive, and convenient diagnosis of mucopolysaccharidoses. Thus, if the diagnostic method of the present invention is performed on newborns, mucopolysaccharidoses can be detected in an early stage after birth, and appropriate enzyme replacement therapy or gene therapy performed in an early stage would restrain development of the pathological conditions of the patient.

In addition to the use in diagnosis of mucopolysaccharidoses, the method of the present invention can also be used to comprehend the therapeutic effect of the aforementioned therapy, to decide on therapeutic options, and to evaluate drug efficacy in the development of pharmaceuticals.

Moreover, the method of the present invention finds utility in biomarker assays performed for identifying GAG-related pathological conditions, such as inflammations associated with arthrosis deformans, chronic articular rheumatism, or diseases accompanied by abnormalities in corneal tissue; carcinomas; and liver diseases.

### BRIEF DESCRIPTION OF THE DRAWINGS

FIG. 1 is a graph showing the relation between mobile phase pH and elution position.

FIG. 2 is a graph showing the relation between salt concentration of the mobile phase and elution position.

FIGS. 3A and 3B provide chromatograms showing peak profiles of mobile phase pH, which affect the separation.

### BEST MODES FOR CARRYING OUT THE INVENTION

No particular limitation is imposed on the biological sample employed in step (1) of the method of the present invention, so long as the sample contains mucopolysaccharides. Examples of the biological sample include plasma, serum, blood, urine, and body fluid. Of these, plasma and serum are particularly preferred.

No particular limitation is imposed on the ultrafiltration filter employed in the present invention, so long as the filter does not allow mucopolysaccharides to pass therethrough, but allow passage of molecules smaller than mucopolysaccharides in molecular weight. Preferably, the filter can isolate molecules having a molecular weight of about 5000. Examples of commercially available ultrafiltration filters which may be employed in the present invention include ULTRAFREET™-MC (BIOMAX-5) (product of MILLIPORE). When an AcroPrep 96 filter plate (10K) (product of PALL Life Sciences) is employed, simultaneous processing can be performed on multiple samples.

No particular limitation is imposed on the GAG-specific enzymes employed in the present invention, so long as the enzymes degrade glycosaminoglycans. Exemplary enzymes are those which act specifically on KS, HS or DS and degrade the same. These enzymes may be employed singly or in combination of two or more species. When the three enzymes; i.e., keratan sulfate degrading enzyme, heparan sulfate degrading enzyme, and dermatan sulfate degrading enzyme, are employed in combination, keratan sulfate, heparan sulfate, and dermatan sulfate are all degraded simultaneously, whereas when one of these enzymes is employed, one or two species of these glycosaminoglycans can be analyzed. Preferred examples of the GAG-degrading enzymes include keratanase, heparitinase, and chondroitinase B. Examples of commercially available GAG-specific enzymes include keratanase, keratanase II, heparitinase, heparitinase I, heparitinase II, heparinase, and chondroitinase B (produced and sold by SEIKAGAKU CORPORATION). As for the HS degrading enzyme, an enzyme having a similar effect, which is commercially available from Sigma Co., may be employed. Of the above-mentioned enzymes, most preferably, the three enzymes of keratanase II, heparitinase, and chondroitinase B are employed in combination, or alternatively, one of these three enzymes is employed.

Enzymatic digestion by the GAG-specific enzyme(s) performed according to the present invention is complete after, for example, 1- to 30-hour digestion at 30 to 40° C. Preferably, enzymatic digestion is performed in a 37° C. incubator for 15 hours.

In one application of the present invention, when CS or HA is a target substance which is desired to be measured, chondroitinase ABC, chondroitinase ACII, or hyaluronidase SD may be used to specifically degrade CS or HA, followed by LC/MS/MS for analysis.

Glycosaminoglycans are degraded to disaccharides through enzymatic digestion using the above-mentioned GAG-specific enzymes. Some abbreviations of disaccharides are provided below.

ΔDiHS-0S: ΔHexA α1→4GlcNAc: 2-acetamido-2-deoxy-4-O-(4-deoxy-α-L-threo-hex-enopyranosyluronic acid)-D-glucose, ΔDiHS-NS: ΔHexA α1→4GlcNS: 2-deoxy-2-sulfamino-4-O-(4-deoxy-α-L-threo-hex-4-enopyranosyluronic acid)-D-glucose, ΔDiHS-6S: ΔHexA α1→4GlcNAc(6S): 2-acetamido-2-deoxy-4-O-(4-deoxy-α-L-threo-hex-4-enopyranosyluronic acid)-6-O-D-glucose, MSD: Galβ1→3GlcNAc(6S), DSD: Gal(6S)β1→3GlcNAc(6S).

The step (1) of the present invention includes (a) a means which comprises filtering a biological sample with an ultrafiltration filter, and digesting the biological sample on the filter with a GAG-specific enzyme, and (b) a means which comprises digesting a biological sample with a GAG-specific enzym, and filtering the digested biological sample with an ultrafiltration filter. The means (b) may be performed, for instance, by drawing a small amount of blood from the ear lobe of a subject, digesting a blood-impregnated filter paper with a GAG-specific enzyme, and filtering the digested substance with an ultrafiltration filter.

The disaccharides which are measurement targets in the present invention are MSD and DSD (degradation products of KS by keratanase II); ΔDiHS-0S, ΔDiHS-NS, and ΔDiHS-6S (degradation products of HS by heparitinase); and ΔDi-4S (degradation products of DS by chondroitinase B).

A digestion product obtained from the above process is centrifuged and the filtrate is injected to LC/MS/MS for analysis of disaccharides. Preferably, centrifugation is performed, for example, at 5000 to 8000×g for 10 to 15 minutes.

No particular limitation is imposed on the analytical column of LC/MS/MS, so long as the column can separate the above-mentioned disaccharides. Examples of the column include a carbon graphite column and a reverse phase HPLC column in which ODS (octadecylsilane) is employed as a stationary phase. For obtaining good resolution, a carbon graphite column is preferred. Examples of commercially available carbon graphite columns include Hypercarb (2.0 mm i.d.×150 mm, 5 μm) (product of Thermo Electron Corp). When a column having a shorter length is employed, retention time of disaccharides can be shortened.

In the present invention, in order to optimize the elution positions of disaccharides, preferably, the mobile phase is an alkaline solution. The alkaline solution is preferably of pH 7 to 11, more preferably pH 8 to 10, still more preferably pH 9 to 10, particularly preferably pH 10, and gradient conditions are preferably established together with an organic solvent. A preferred salt for adjusting pH to fall within an alkaline range is aqueous ammonia or an ammonium salt. Exemplary aqueous ammonium salt solutions include aqueous ammonium bicarbonate solution, aqueous ammonium formate solution, and aqueous ammonium acetate solution, with aqueous ammonium bicarbonate solution being preferred. For attaining good elution positions, the salt concentration of any of the above solutions is preferably 3 to 100 mmol/L, more preferably 3 to 50 mmol/L, even more preferably 10 mmol/L. Examples of the organic solvent include acetonitrile, methanol, ethanol, and 2-propanol. Most preferably, gradient conditions are conducted using a solution of pH 10 prepared through addition of 28% aqueous ammonia to 10 mmol/L ammonium bicarbonate solution (10 mmol/L ammonium bicarbonate buffer (pH 10)) and acetonitrile.

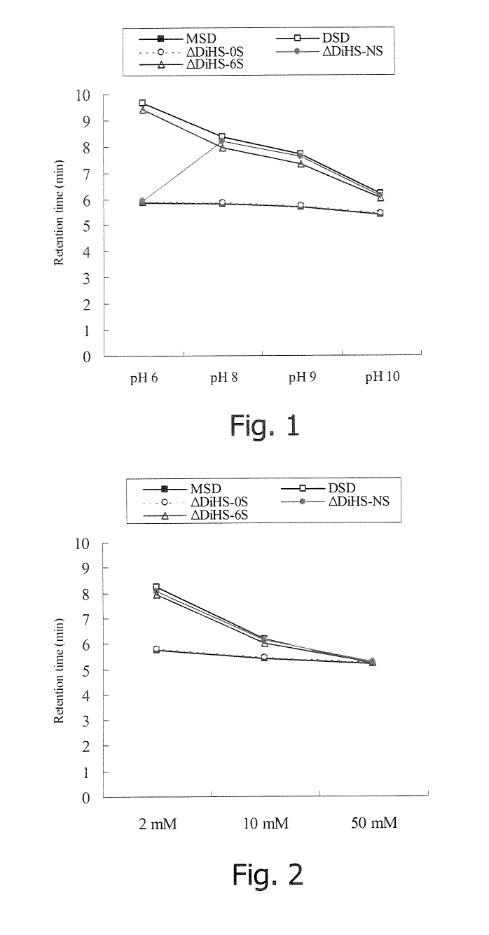

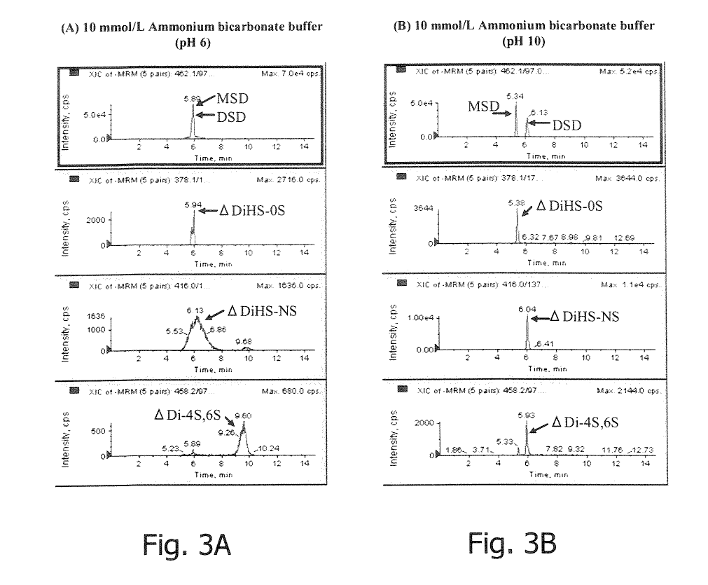

As shown in FIGS. 1 and 2, when the pH and the salt concentration of the mobile phase are regulated, GAG-derived disaccharides can be eluted at elution positions (i.e., optimal retention times) that are optimal for the MS analysis. In addition, as shown in FIGS. 3A and 3B, through maneuvering the pH of the mobile phase, the peak shape was improved significantly. Thus, this approach enables retention time regulation of saccharides, which has otherwise been very difficult according to conventional methods.

Through the above-described sub-steps in step (1), the GAG level and the disaccharide composition of a biological sample can be obtained. In step (2), on the basis of the data obtained in step (1), diagnosis of mucopolysaccharidosis can be rendered, and moreover, the type of mucopolysaccharidosis can be determined. Furthermore, effect of a therapy of mucopolysaccharidosis can be assessed. Table 1 shows a classification of mucopolysaccharidoses.

TABLE 1

| | Class name | Lacking enzyme |
|---|---|---|
| IH | Hurler syndrome | α-L-iduronidase |
| IS | Scheie syndrome | α-L-iduronidase |
| IH/S | Hurler-Scheie syndrome | α-L-iduronidase |
| IIA | Hunter syndrome, severe type | sulfoiduronate sulfatase |
| IIB | Hunter syndrome, mild type | sulfoiduronate sulfatase |
| IIIA | Sanfilippo syndrome A | heparan sulfate N-sulfatase |
| IIIB | Sanfilippo syndrome B | N-acetyl-α-D- |
| | | glucosaminidase |
| IIIC | Sanfilippo syndrome C | acetyl-CoA-α-glucosaminide |
| | | N-acetyltransferase |
| IIID | Sanfilippo syndrome D | N-acetylgiucosamine-6- |
| | | sulfatase |
| IVA | Morquio syndrome A | N-acetylgalactosamine-6- |
| | | sulfatase |
| IVB | Morquio syndrome B | β-galactosidase |
| VIA | Maroteaux-Lamy syndrome, | N-acetylgalactosamine-4- |
| | severe type | sulfatase |
| VIB | Maroteaux-Lamy syndrome, | N-acetylgalactosamine-4- |
| | mild type | sulfatase |
| VII | β-glucuronidase deficiency | β-glucuronidase |

### EXAMPLES

The present invention will next be described in detail by way of examples, which should not be construed as limiting the invention thereto.

### Example 1

In order to check whether the assay method of the present invention provides a successful screening on plasma or serum samples, the following experiment was performed using plasma samples from mucopolysaccharidosis patients and control plasma samples (human).

Pretreatment of a plasma or serum sample:
1) Add a plasma or serum sample (0.01 mL) to ULTRAFREE™-MC (BIOMAX-5);
2) Centrifuge at 4,000×g for 15 minutes;
3) Replace the collection tube in ULTRAFREE™-MC (BIOMAX-5) by a new tube;
4) Add a 50-μg/mL aqueous chondrosine solution (0.02 mL) (produced and sold by SEIKAGAKU CORPORATION) as an internal standard substance onto the filter (note: throughout the procedures, water should be purified water);
5) Add 50-mmol/L Tris-HCl buffer (0.02 mL, pH 7) onto the filter;
6) Add an enzyme mixture solution (0.02 mL) containing keratanase II, heparitinase, and chondroitinase B (2 mU each) onto the filter;
7) Mix the resultant mixture using a vortex mixer for about ten seconds;
8) Incubate the mixture at 37° C. for 15 hours;
9) Centrifuge the resultant mixture at 8,000×g for 15 minutes;
10) Add water (0.02 mL) to the filtrate;
11) Mix the resultant mixture using a vortex mixer for about 10 seconds; and
12) Transfer the-thus obtained liquid sample into an injection vial for an autosampler.

Pretreatment of a sample for producing a calibration curve:
1) KS standard solutions: Bovine-cornea-derived KS (produced and sold by SEIKAGAKU CORPORATION) is employed.
Concentrations are shown in Table 2.
2) HS standard solutions: An unsaturated heparan/heparin-disaccharide kit (H kit) (produced and sold by SEIKAGAKU CORPORATION) is employed. Aqueous solutions each containing ΔDiHS-0S, ΔDiHS-6S, and ΔDiHS-NS are prepared.
Concentrations are shown in Table 3.
3) Add an aliquot (0.01 mL) of each of the above-prepared KS standard solutions and an aliquot (0.01 mL) of each of the above-prepared HS standard solutions to ULTRAFREE™-MC (BIOMAX-5).
4) Add an 50-pg/mL aqueous solution (0.02 mL) of chondrosine (produced and sold by SEIKAGAKU CORPORATION) as an internal standard substance onto the filter.
5) Adding 50-mmol/L Tris-HCl buffer (0.02 mL, pH 7) on the filter.
6) Add an enzyme-mixed aqueous solution (0.02 mL) containing keratanase II, heparitinase, and chondroitinase B (2 mU each) onto the filter.
7) Mix the resultant mixture by use of a vortex mixer for about ten seconds.
8) Incubate the mixture at 37° C. for 15 hours.
9) Centrifuge the resultant mixture at 8,000×g for 15 minutes. 10) Add blank plasma or blank serum to ULTRAFREE™-MC (BIOMAX-5) then centrifuge at 8,000×g for 15 minutes, to thereby prepare a blank filtrate.
11) Add the thus-prepared blank filtrate (0.01 mL) to the filtrate obtained in step 9).
12) Mix the resultant mixture using a vortex mixer for about 10 seconds.

13) Transfer the-thus obtained liquid sample into an injection vial for an autosampler.

TABLE 2
Concentration of standard solution (KS)

(Unit: μg/mL)

| | S7 | S6 | S5 | S4 | S3 | S2 | S1 |
|---|---|---|---|---|---|---|---|
| MSD | 7.1 | 3.6 | 2.8 | 1.4 | 0.71 | 0.36 | 0.14 |
| DSD | 2.9 | 1.5 | 1.2 | 0.58 | 0.29 | 0.15 | 0.058 |
| Total | 10 | 5 | 4 | 2 | 1 | 0.5 | 0.2 |

TABLE 3
Concentration of standard solution (HS)

| | | | | (Unit: ng/mL) | | | |
|---|---|---|---|---|---|---|---|
| | S7 | S6 | S5 | S4 | S3 | S2 | S1 |
| ΔDiHS-0S | 1000 | 500 | 200 | 100 | 50 | 20 | 10 |
| ΔDiHS-NS | 500 | 250 | 100 | 50 | 25 | 10 | 5 |
| ΔDiHS-6S | 1000 | 500 | 200 | 100 | 50 | 20 | 10 |

The LS/MS/MS apparatus employed are as follows:

HPLC apparatus: HP1100 system (Agilent Technology Inc.) (Palo Alto, Calif., USA), autosampler: HTC PAL (CTC Analytics Inc.) (Zwingen, Switzerland), mass spectrometer: API 4000 (Applied Biosystems Inc.) (Lincoln Centre Drive Foster City, Calif., USA).

The HPLC conditions employed are as follows.

Analytical column: Hypercarb (2.0 mm i.d.×150 mm, 5 μm) (Thermo Electron Corp.) (Waltham, Mass., USA), mobile phase: (A) 10 mmol/L Ammonium bicarbonate buffer (pH 10), (B) Acetonitrile, gradient conditions: [Time(min)/B(%)]; [0/0]→[0.9/0]→[1.0/30]→[6.0/30]→[6.1/0]→[8.0/0], rate flow: 0.2 mL/min, column temperature 45° C., the volume of injection into an autosampler: 0.01 mL.

The MS/MS conditions employed are as follows.

Ionization method: turbo ionspray, detection mode: multiple reaction monitoring (MRM)-negative mode, turbospray temperature: 650° C., monitoring ion (CID energy): Galβ¹⁻³GlcNAc(6S)m/z 462.1-m/z 97.0 (CID: −80 eV); Gal(6S)β¹⁻³GlcNAc(6S)m/z 462.1-m/z 97.0 (CID: −80 eV); ΔDiHS-0S m/z 378.1-m/z 174.9 (CID: −22 eV); ΔDiHS-NS m/z 416.0-m/z 137.9 (CID: −34 eV); ΔDiHS-6S m/z 458.2-m/z 97.1 (CID: −52 eV); I.S.m/z 354.0-m/z 113.0 (CID: −22 eV).

For calculation of concentrations, a linear first-order regression equation was established using concentrations on the calibration curve, peak area ratio (“peak area of the standard substance of each analyte”/“peak area of an internal standard substance”), and the method of least squares. A weighting of 1/“calibration curve concentration” was used for curve fit.

Three different control serum samples were measured for three days (N=5). The results are shown in Tables 4 and 5.

TABLE 4

| | | MSD | | | DSD | | |
|---|---|---|---|---|---|---|---|
| | Replicates | No. 1 | No. 2 | No. 3 | No. 1 | No. 2 | No. 3 |
| Batch 1 | n1 | 0.97 | 0.52 | 0.60 | 0.34 | 0.18 | 0.21 |
| | n2 | 0.96 | 0.51 | 0.64 | 0.36 | 0.19 | 0.22 |
| | n3 | 1.1 | 0.54 | 0.58 | 0.37 | 0.18 | 0.19 |
| | n4 | 0.97 | 0.54 | 0.62 | 0.35 | 0.18 | 0.21 |
| | n5 | 1.1 | 0.55 | 0.61 | 0.36 | 0.19 | 0.21 |
| | Mean | 1.0 | 0.53 | 0.61 | 0.36 | 0.18 | 0.21 |
| | SB | 0.073 | 0.016 | 0.022 | 0.0114 | 0.0055 | 0.0110 |
| | CV % | 7.2 | 3.1 | 3.7 | 3.2 | 3.0 | 5.3 |
| Batch 2 | n1 | 0.94 | 0.59 | 0.70 | 0.35 | 0.19 | 0.22 |
| | n2 | 0.93 | 0.59 | 0.67 | 0.37 | 0.18 | 0.21 |
| | n3 | 1.1 | 0.54 | 0.65 | 0.34 | 0.18 | 0.21 |
| | n4 | 1.0 | 0.58 | 0.65 | 0.35 | 0.19 | 0.20 |
| | n5 | 1.1 | 0.58 | 0.63 | 0.35 | 0.18 | 0.20 |
| | Mean | 1.01 | 0.58 | 0.66 | 0.35 | 0.18 | 0.21 |
| | SD | 0.083 | 0.021 | 0.026 | 0.0110 | 0.0055 | 0.0084 |
| | CV % | 8.2 | 3.6 | 4.0 | 3.1 | 3.0 | 4.0 |
| Batch 3 | n1 | 1.0 | 0.52 | 0.62 | 0.35 | 0.18 | 0.19 |
| | n2 | 1.0 | 0.52 | 0.63 | 0.36 | 0.17 | 0.19 |
| | n3 | 0.96 | 0.56 | 0.60 | 0.35 | 0.17 | 0.19 |
| | n4 | 1.1 | 0.52 | 0.61 | 0.37 | 0.17 | 0.19 |
| | n5 | 0.96 | 0.53 | 0.62 | 0.35 | 0.17 | 0.19 |
| | Mean | 1.00 | 0.53 | 0.62 | 0.36 | 0.17 | 0.19 |
| | SD | 0.057 | 0.017 | 0.011 | 0.0089 | 0.0045 | 0.0000 |
| | CV % | 5.7 | 3.3 | 1.9 | 2.5 | 2.6 | 0.0 |
| Overall | Mean | 1.01 | 0.55 | 0.63 | 0.35 | 0.18 | 0.20 |
| (N = 15) | SD | 0.067 | 0.028 | 0.030 | 0.010 | 0.0076 | 0.0115 |
| | CV % | 6.6 | 5.1 | 4.8 | 2.8 | 4.2 | 5.7 |

TABLE 5

| | | ΔDiHS-0S | | | ΔDiHS-NS | | | ΔDi-4S, 6S* | | |
|---|---|---|---|---|---|---|---|---|---|---|
| | Replicates | No. 1 | No. 2 | No. 3 | No. 1 | No. 2 | No. 3 | No. 1 | No. 2 | No. 3 |
| Batch 1 | n1 | 59 | 53 | 72 | 19 | 15 | 19 | 54 | 17 | 18 |
| | n2 | 58 | 51 | 70 | 19 | 16 | 18 | 53 | 17 | 24 |
| | n3 | 61 | 55 | 73 | 22 | 16 | 21 | 53 | 19 | 23 |
| | n4 | 60 | 52 | 77 | 19 | 15 | 20 | 52 | 19 | 28 |
| | n5 | 66 | 53 | 74 | 20 | 17 | 18 | 53 | 17 | 26 |
| | Mean | 61 | 53 | 73 | 20 | 16 | 19 | 53 | 18 | 24 |
| | SD | 3.1 | 1.5 | 2.6 | 1.3 | 0.84 | 1.3 | 0.71 | 1.1 | 3.8 |
| | CV % | 5.1 | 2.8 | 3.5 | 6.6 | 5.3 | 6.8 | 1.3 | 6.2 | 15.8 |
| Batch 2 | n1 | 54 | 49 | 74 | 20 | 16 | 19 | 64 | 25 | 25 |
| | n2 | 57 | 49 | 74 | 22 | 16 | 19 | 60 | 18 | 23 |
| | n3 | 61 | 48 | 70 | 21 | 14 | 20 | 66 | 19 | 24 |
| | n4 | 55 | 49 | 70 | 22 | 14 | 19 | 51 | 22 | 22 |
| | n5 | 63 | 50 | 71 | 19 | 14 | 19 | 57 | 19 | 26 |
| | Mean | 58 | 49 | 72 | 21 | 15 | 19 | 60 | 21 | 24 |
| | SD | 3.9 | 0.71 | 2.0 | 1.3 | 1.1 | 0.45 | 5.9 | 2.9 | 1.6 |
| | CV % | 6.7 | 1.4 | 2.9 | 6.3 | 7.4 | 2.3 | 10.0 | 14.0 | 6.6 |
| Batch 3 | n1 | 58 | 48 | 62 | 20 | 14 | 18 | 60 | 15 | 19 |
| | n2 | 59 | 48 | 71 | 22 | 15 | 19 | 67 | 18 | 21 |
| | n3 | 59 | 49 | 68 | 21 | 14 | 16 | 75 | 18 | 20 |
| | n4 | 61 | 52 | 67 | 19 | 15 | 18 | 81 | 18 | 21 |
| | n5 | 57 | 49 | 67 | 18 | 15 | 19 | 66 | 15 | 20 |
| | Mean | 59 | 49 | 67 | 20 | 15 | 18 | 70 | 17 | 20 |
| | SD | 1.5 | 1.6 | 3.2 | 1.6 | 0.55 | 1.2 | 8.2 | 1.6 | 0.84 |
| | CV % | 2.5 | 3.3 | 4.8 | 7.9 | 3.8 | 6.8 | 11.8 | 9.8 | 4.1 |
| Overall | Mean | 59 | 50 | 71 | 20 | 15 | 19 | 61 | 18 | 23 |
| (N = 15) | SD | 3.0 | 2.2 | 3.7 | 1.4 | 0.96 | 1.1 | 9.0 | 2.5 | 2.9 |
| | CV % | 5.1 | 4.4 | 5.2 | 6.8 | 6.4 | 6.1 | 14.8 | 13.6 | 12.7 |

*ΔDi-4S, 6S: These disaccharides were produced from DS and HS(6S).

Three different control plasma samples were measured for one day (N=5). The results are shown in Tables 6 and 7.

TABLE 6

| | | MSD | | | DSD | | |
|---|---|---|---|---|---|---|---|
| | Replicates | No. 1 | No. 2 | No. 3 | No. 1 | No. 2 | No. 3 |
| Concen- | n1 | 0.40 | 0.32 | 0.34 | 0.12 | 0.13 | 0.11 |
| tration | n2 | 0.38 | 0.34 | 0.38 | 0.12 | 0.12 | 0.11 |
| (μg/mL) | n3 | 0.35 | 0.32 | 0.33 | 0.12 | 0.12 | 0.10 |
| | n4 | 0.37 | 0.33 | 0.30 | 0.12 | 0.12 | 0.095 |
| | n5 | 0.37 | 0.33 | 0.33 | 0.11 | 0.12 | 0.11 |
| Mean | | 0.37 | 0.33 | 0.34 | 0.12 | 0.12 | 0.11 |
| SD | | 0.018 | 0.008 | 0.029 | 0.0045 | 0.0045 | 0.0071 |
| CV % | | 4.9 | 2.6 | 8.6 | 3.8 | 3.7 | 6.7 |

TABLE 7

| | Repli- | ΔDiHS-0S | | | ΔDiHS-NS | | | ΔDi-4S, -6S* | | |
|---|---|---|---|---|---|---|---|---|---|---|
| | cates | No. 1 | No. 2 | No. 3 | No. 1 | No. 2 | No. 3 | No. 1 | No. 2 | No.3 |
| Concen- | n1 | 91 | 64 | 53 | 13 | 14 | 12 | 54 | 140 | 90 |
| tration | n2 | 110 | 67 | 52 | 13 | 14 | 12 | 55 | 180 | 130 |
| (μg/mL) | n3 | 100 | 58 | 50 | 15 | 11 | 12 | 55 | 170 | 91 |
| | n4 | 96 | 54 | 47 | 15 | 13 | 13 | 55 | 170 | 97 |
| | n5 | 83 | 53 | 53 | 15 | 14 | 12 | 68 | 180 | 130 |
| Mean | | 96 | 59 | 51 | 14 | 13 | 12 | 57 | 168 | 108 |
| SD | | 10.1 | 6.1 | 2.5 | 1.1 | 1.3 | 0.4 | 5.9 | 16.4 | 20.6 |
| CV % | | 10.5 | 10.4 | 5.0 | 7.7 | 9.9 | 3.7 | 10.4 | 9.8 | 19.2 |

*ΔDi-4S, 6S: These disaccharides were produced from DS and HS(6S).

In Tables 5 and 7, the concentration data of ΔDi-4S, 6S represent a total concentration of DS-derived ΔDi-4S and HS-derived ΔDiHS-6S.

As is clear from Tables 4 to 7, the method of the present invention is an accurate, precise analytical method.

The results of measurement on plasma samples from mucopolysaccharidosis patients and control plasma samples are shown in Tables 8 and 9.

TABLE 8

| | | | Concentarations | | | Composition | |
|---|---|---|---|---|---|---|---|
| Sample | | Age | (μg/mL) | | | (%) | |
| No. | Categoly | (years) | MSD | DSD | Total | MSD | DSD |
| 1 | MPS I | 1.2 | 3.1 | 0.50 | 3.6 | 86 | 14 |
| 2 | MPS I | 0.1 | 4.6 | 1.0 | 5.6 | 82 | 18 |
| 3 | MPS II | 15 | 4.0 | 0.76 | 4.8 | 84 | 16 |
| 4 | MPS II | 19 | 4.5 | 0.86 | 5.4 | 84 | 16 |
| 5 | MPS II | 19 | 5.0 | 1.3 | 6.3 | 79 | 21 |
| 6 | MPS IIIA | 4.5 | 2.4 | 0.72 | 3.1 | 77 | 23 |
| 7 | MPS IIIA | 0.7 | 2.6 | 0.51 | 3.1 | 84 | 16 |
| 8 | MPS IIIB | 4.5 | 2.2 | 0.40 | 2.6 | 85 | 15 |
| 9 | MPS IIIB | 6.5 | 2.4 | 0.80 | 3.2 | 75 | 25 |
| 10 | MPS IIIC | 6 | 3.0 | 0.83 | 3.8 | 78 | 22 |
| 11 | MPS IV | 3.3 | 7.0 | 2.4 | 9.4 | 74 | 26 |
| 12 | MPS IV | 3.5 | 3.7 | 1.1 | 4.8 | 77 | 23 |
| 13 | MPS VI | NA | 1.9 | 0.32 | 2.2 | 86 | 14 |
| 14 | MPS VI | 6.7 | 4.0 | 1.3 | 5.3 | 75 | 25 |
| 15 | MPS VII | 7 | 1.3 | 0.28 | 1.6 | 82 | 18 |
| 16 | MPS VII | 0.5 | 2.6 | 0.63 | 3.2 | 80 | 20 |
| 17 | Control | 43 | 0.76 | 0.16 | 0.92 | 83 | 17 |
| 18 | Control | 14 | 0.96 | 0.22 | 1.2 | 81 | 19 |
| 19 | Control | 51 | 0.89 | 0.29 | 1.2 | 75 | 25 |
| 20 | Control | 30 | 0.60 | 0.18 | 0.78 | 77 | 23 |
| 21 | Control | 34 | 0.76 | 0.26 | 1.0 | 75 | 25 |
| 22 | Control | 12 | 2.2 | 0.45 | 2.7 | 83 | 17 |
| 23 | Control | 4 | 1.1 | 0.36 | 1.5 | 75 | 25 |
| 24 | Control | 1 | 1.8 | 0.36 | 2.2 | 83 | 17 |
| 25 | Control | 14 | 2.2 | 0.71 | 2.9 | 76 | 24 |
| 26 | Control | 23 | 0.46 | 0.13 | 0.59 | 78 | 22 |
| 27 | Control | 26 | 0.73 | 0.21 | 0.94 | 78 | 22 |
| 28 | Control | 31 | 0.43 | 0.13 | 0.56 | 77 | 23 |
| 29 | Control | 36 | 1.6 | 0.38 | 2.0 | 81 | 19 |

NA: Not available.

TABLE 9

| | | | Concentarations | | | | Composition | | |
|---|---|---|---|---|---|---|---|---|---|
| Sample | | Age | (ng/mL) | | | | (%) | | |
| No. | Categoly | (years) | ΔDiHS-0S | ΔDiHS-NS | ΔDi-4S, -6S* | Total | ΔDiHS-0S | ΔDiHS-NS | ΔDi-4S, -6S* |
| 1 | MPS I | 1.2 | 1200 | 250 | 590 | 2040 | 59 | 12 | 29 |
| 2 | MPS I | 0.1 | 8500 | 3300 | 12000 | 23800 | 36 | 14 | 50 |
| 3 | MPS II | 15 | 850 | 190 | 230 | 1270 | 67 | 15 | 18 |
| 4 | MPS II | 19 | 670 | 160 | 320 | 1150 | 58 | 14 | 28 |
| 5 | MPS II | 19 | 1100 | 270 | 1800 | 3170 | 35 | 9 | 57 |
| 6 | MPS IIIA | 4.5 | 1400 | 320 | 68 | 1788 | 78 | 18 | 4 |
| 7 | MPS IIIA | 0.7 | 2900 | 590 | 640 | 4130 | 70 | 14 | 15 |
| 8 | MPS IIIB | 4.5 | 1200 | 270 | 61 | 1531 | 78 | 18 | 4 |
| 9 | MPS IIIB | 6.5 | 2600 | 770 | 530 | 3900 | 67 | 20 | 14 |
| 10 | MPS IIIC | 6 | 1200 | 280 | 470 | 1950 | 62 | 14 | 24 |
| 11 | MPS IV | 3.3 | 520 | 90 | 700 | 1310 | 40 | 7 | 53 |
| 12 | MPS IV | 3.5 | 360 | 59 | 780 | 1199 | 30 | 5 | 65 |
| 13 | MPS VI | NA | 340 | 73 | 590 | 1003 | 34 | 7 | 59 |
| 14 | MPS VI | 6.7 | 340 | 62 | 1400 | 1802 | 19 | 3 | 78 |
| 15 | MPS VII | 7 | 210 | 19 | 33 | 262 | 80 | 7 | 13 |
| 16 | MPS VII | 0.5 | 980 | 180 | 700 | 1860 | 53 | 10 | 38 |
| 17 | Control | 43 | 120 | 20 | 88 | 228 | 53 | 9 | 39 |
| 18 | Control | 14 | 130 | 23 | 240 | 393 | 33 | 6 | 61 |
| 19 | Control | 51 | 120 | 24 | 260 | 404 | 30 | 6 | 64 |
| 20 | Control | 30 | 130 | 26 | 260 | 416 | 31 | 6 | 63 |
| 21 | Control | 34 | 130 | 24 | 260 | 414 | 31 | 6 | 63 |
| 22 | Control | 12 | 150 | 25 | 170 | 345 | 43 | 7 | 49 |
| 23 | Control | 1 | 290 | 46 | 320 | 656 | 44 | 7 | 49 |
| 24 | Control | 14 | 350 | 55 | 350 | 755 | 46 | 7 | 46 |
| 25 | Control | 31 | 220 | 22 | 69 | 311 | 71 | 7 | 22 |
| 26 | Control | 36 | 470 | 78 | 340 | 888 | 53 | 9 | 38 |

*ΔDi-4S, 6S: These disaccharides were produced from DS and HS(6S).
NA: Not available.

As is clear from Tables 8 and 9, the method of the present invention has been found to be useful in an assay of a clinical sample and also in screening. A mucopolysaccharidosis type IV case (No. 11 in Table 8) showed a high KS concentration. Also, mucopolysaccharidosis type I, II, and III cases (Nos. 1 to 10 in Table 9) showed high values of HS-derived ΔDiHS-0S concentration and HS-derived ΔDiHS-NS concentration. Moreover, a mucopolysaccharidosis type VI case (No. 14 in Table 9) showed a high value of DS-derived ΔDi-4S,6S concentration.

In cases where ΔDi-4S,6S level was high, DS or HS was also found to be high. However, when ΔDi-4S,6S has a high compositional proportion of disaccharides, a high value of ΔDi-4S,6S reflects a high DS value. In other words, the method of the present invention, which can provide analyses of concentration data of respective disaccharides and compositional proportions, is very useful for attaining a detailed analysis.

As described above, with the present method, KS, HS, and DS levels can be analyzed simultaneously. If some correlation is identified in future research between age, pathological conditions, etc. of a patient and KS, HS, and DS levels, it is believed that a single assay provides separate, simultaneous diagnosis of different types of mucopolysaccharidoses.

### Example 2

In order to check whether the assay method of the present invention provides a successful screening on urine samples, the following experiment was performed using urine samples from mucopolysaccharidosis patients and control urine samples (human).

Pretreatment of a urine sample:
1) Add a urine sample (0.01 mL) to ULTRAFREE™-MC (BIOMAX-5);
2) Centrifuge at 4,000×g for 15 minutes;
3) Replace the collection tube in ULTRAFREE™-MC (BIOMAX-5) by a new tube;
4) Add a 50μg/mL aqueous chondrosine solution (0.02 mL) (produced and sold by SEIKAGAKU CORPORATION) as an internal standard substance onto the filter;
5) Add 50-mmol/L Tris-HCl buffer (0.02 mL, pH 7) onto the filter;
6) Add an enzyme mixture solution (0.02 mL) containing keratanase II, heparitinase, and chondroitinase B (2 mU each) onto the filter;
7) Mix the resultant mixture using a vortex mixer for about ten seconds;
8) Incubate the mixture at 37° C. for 15 hours;
9) Centrifuge the resultant mixture at 8,000×g for 15 minutes;
10) Add water (0.02 mL) to the filtrate;
11) Mix the resultant mixture using a vortex mixer for about 10 seconds; and
12) Transfer the-thus obtained liquid sample into an injection vial for an autosampler.

Pretreatment of a sample for producing a calibration curve: 1) KS standard solutions: Bovine-cornea-derived KS (produced and sold by SEIKAGAKU CORPORATION) is employed.

Concentrations are shown in Table 10.

2) HS standard solutions: An unsaturated heparan/heparin-disaccharide kit (H kit) (produced and sold by SEIKAGAKU CORPORATION) is employed. Aqueous solutions each containing ΔDiHS-0S, ΔDiHS-6S, and ΔDiHS-NS are prepared.
   Concentrations are shown in Table 11.
3) Add an aliquot (0.01 mL) of each of the above-prepared KS standard solutions and an aliquot (0.02 mL) of each of the above-prepared HS standard solutions to ULTRAFREE™-MC (BIOMAX-5).
4) Add an 50-μg/mL aqueous solution (0.02 mL) of chondrosine (produced and sold by SEIKAGAKU CORPORATION) as an internal standard substance onto the filter.
5) Adding 50-mmol/L Tris-HCl buffer (0.02 mL, pH 7) on the filter.
6) Add an enzyme-mixed aqueous solution (0.02 mL) containing keratanase II, heparitinase, and chondroitinase B (2 mU each) onto the filter.
7) Mix the resultant mixture by use of a vortex mixer for about ten seconds.
8) Incubate the mixture at 37° C. for 15 hours.
9) Centrifuge the resultant mixture at 8,000×g for 15 minutes.
10) Allow a blank urine sample to pass through a stationery column, Bond Elute SAX column (500 mg/3 mL), to thereby prepare a blank solution.
11) Add the thus-prepared blank solution (0.01 mL) to the filtrate obtained in step 9).
12) Mix the resultant mixture using a vortex mixer for about 10 seconds.

13) Transfer the-thus obtained liquid sample into an injection vial for an autosampler.

TABLE 10
Concentration of standard solution (KS)

| | | | | | (Unit: μg/mL) | | |
|---|---|---|---|---|---|---|---|
| | S7 | S6 | S5 | S4 | S3 | S2 | S1 |
| MSD | 7.1 | 3.6 | 2.8 | 1.4 | 0.71 | 0.36 | 0.14 |
| DSD | 2.9 | 1.5 | 1.2 | 0.58 | 0.29 | 0.15 | 0.058 |
| Total | 10 | 5 | 4 | 2 | 1 | 0.5 | 0.2 |

TABLE 11
Concentration of standard solution (HS)

| | | | | (Unit: ng/mL) | | |
|---|---|---|---|---|---|---|
| | S6 | S5 | S4 | S3 | S2 | S1 |
| ΔDiHS-0S | 2500 | 1250 | 500 | 250 | 100 | 50 |
| ΔDiHS-NS | 1250 | 625 | 250 | 125 | 50 | 25 |
| ΔDIHS-6S | 625 | 313 | 125 | 63 | 25 | 13 |

In the analysis of urine samples, LC/MS/MS conditions employed and concentration calculation method are the same as those used for the analyses of plasma and serum samples.

Three different control urine samples were measured for three days (N-5). The results are shown in Tables 12 and 13.

TABLE 12

| | | MSD | | | DSD | | |
|---|---|---|---|---|---|---|---|
| | Replicates | No. 1 | No. 2 | No. 3 | No. 1 | No. 2 | No. 3 |
| Batch 1 | n1 | 1.3 | 0.97 | 1.5 | 0.65 | 0.47 | 0.98 |
| | n2 | 1.1 | 1.1 | 1.5 | 0.54 | 0.52 | 0.98 |
| | n3 | 1.2 | 1.1 | 1.6 | 0.62 | 0.49 | 0.96 |
| | n4 | 1.2 | 1.0 | 1.5 | 0.58 | 0.50 | 0.94 |
| | n5 | 1.2 | 1.1 | 1.6 | 0.62 | 0.50 | 1.1 |
| | Mean | 1.2 | 1.1 | 1.5 | 0.60 | 0.50 | 1.0 |
| | SD | 0.071 | 0.064 | 0.055 | 0.043 | 0.018 | 0.063 |
| | CV % | 5.9 | 6.1 | 3.6 | 7.1 | 3.7 | 6.3 |
| Batch 2 | n1 | 1.3 | 1.1 | 1.6 | 0.63 | 0.50 | 1.0 |
| | n2 | 1.3 | 1.1 | 1.6 | 0.72 | 0.50 | 1.1 |
| | n3 | 1.2 | 1.2 | 1.6 | 0.55 | 0.52 | 1.1 |
| | n4 | 1.3 | 1.1 | 1.6 | 0.66 | 0.49 | 1.0 |
| | n5 | 1.2 | 1.2 | 1.6 | 0.54 | 0.53 | 1.1 |
| | Mean | 1.3 | 1.1 | 1.6 | 0.62 | 0.51 | 1.1 |
| | SD | 0.055 | 0.055 | 0.000 | 0.076 | 0.016 | 0.055 |
| | CV % | 4.3 | 4.8 | 0.0 | 12.2 | 3.2 | 5.2 |
| Batch 3 | n1 | 1.4 | 1.2 | 1.8 | 0.65 | 0.47 | 1.0 |
| | n2 | 1.5 | 1.2 | 1.8 | 0.71 | 0.47 | 1.1 |
| | n3 | 1.4 | 1.3 | 1.8 | 0.51 | 0.54 | 1.1 |
| | n4 | 1.5 | 1.2 | 1.8 | 0.66 | 0.49 | 1.0 |
| | n5 | 1.3 | 1.2 | 1.9 | 0.52 | 0.52 | 1.1 |
| | Mean | 1.4 | 1.2 | 1.8 | 0.61 | 0.50 | 1.1 |
| | SD | 0.084 | 0.045 | 0.045 | 0.090 | 0.031 | 0.055 |
| | CV % | 5.9 | 3.7 | 2.5 | 14.7 | 6.3 | 5.2 |
| Overall | Mean | 1.3 | 1.1 | 1.7 | 0.61 | 0.50 | 1.0 |
| (N = 15) | SD | 0.12 | 0.087 | 0.13 | 0.067 | 0.022 | 0.063 |
| | CV % | 9.0 | 7.6 | 7.9 | 11.0 | 4.4 | 6.0 |

TABLE 13

| | | ΔDiHS-0S | | | ΔDiHS-NS | | | ΔDi-4S, 6S* | | |
|---|---|---|---|---|---|---|---|---|---|---|
| | Replicates | No. 1 | No. 2 | No. 3 | No. 1 | No. 2 | No. 3 | No. 1 | No. 2 | No. 3 |
| Batch 1 | n1 | 1100 | 880 | 1800 | 430 | 340 | 1100 | 3100 | 2000 | 6100 |
| | n2 | 900 | 920 | 1900 | 350 | 370 | 1100 | 2400 | 2200 | 6700 |
| | n3 | 980 | 890 | 1800 | 450 | 370 | 1100 | 2900 | 2200 | 7000 |
| | n4 | 1000 | 890 | 1800 | 410 | 350 | 1100 | 3100 | 2100 | 6300 |
| | n5 | 1000 | 880 | 1800 | 450 | 360 | 1100 | 3100 | 2200 | 6900 |
| | Mean | 996 | 892 | 1820 | 418 | 358 | 1100 | 2920 | 2140 | 6600 |
| | SD | 71 | 16 | 45 | 41 | 13 | 0 | 303 | 89 | 387 |
| | CV % | 7.2 | 1.8 | 2.5 | 9.9 | 3.6 | 0.0 | 10.4 | 4.2 | 5.9 |
| Batch 2 | n1 | 990 | 810 | 1800 | 450 | 330 | 980 | 3200 | 2300 | 6400 |
| | n2 | 1100 | 820 | 1800 | 490 | 330 | 1000 | 2900 | 2200 | 6600 |
| | n3 | 890 | 930 | 1900 | 410 | 360 | 1100 | 3000 | 2400 | 6300 |
| | n4 | 1000 | 790 | 1800 | 460 | 310 | 1000 | 3100 | 2100 | 6500 |
| | n5 | 900 | 910 | 1800 | 420 | 330 | 1000 | 2900 | 2300 | 6300 |
| | Mean | 976 | 852 | 1820 | 446 | 332 | 1016 | 3020 | 2260 | 6420 |
| | SD | 86 | 63 | 45 | 32 | 18 | 48 | 130 | 114 | 130 |
| | CV % | 8.8 | 7.4 | 2.5 | 7.2 | 5.4 | 4.7 | 4.3 | 5.0 | 2.0 |
| Batch 3 | n1 | 1100 | 990 | 2000 | 460 | 350 | 1100 | 3100 | 2300 | 6500 |
| | n2 | 1200 | 830 | 2000 | 500 | 360 | 1100 | 3200 | 2000 | 6900 |
| | n3 | 890 | 810 | 2000 | 390 | 390 | 1200 | 2600 | 2600 | 6500 |
| | n4 | 1100 | 940 | 1900 | 480 | 340 | 1100 | 3300 | 2000 | 7000 |
| | n5 | 990 | 790 | 2000 | 420 | 330 | 1100 | 2800 | 2300 | 6400 |
| | Mean | 1056 | 872 | 1980 | 450 | 354 | 1120 | 3000 | 2240 | 6660 |
| | SD | 119 | 88 | 45 | 45 | 23 | 45 | 292 | 251 | 270 |
| | CV % | 11.3 | 10.1 | 2.3 | 9.9 | 6.5 | 4.0 | 9.7 | 11.2 | 4.1 |
| Overall | Mean | 1009 | 872 | 1873 | 438 | 348 | 1079 | 2980 | 2213 | 6560 |
| (N = 15) | SD | 94 | 61 | 88 | 40 | 21 | 58 | 240 | 164 | 282 |
| | CV % | 9.3 | 7.0 | 4.7 | 9.1 | 6.0 | 5.4 | 8.0 | 7.4 | 4.3 |

*ΔDi-4S, 6S: These disaccharides were produced from DS and HS(6S).

As is apparent from Tables 12 and 13, the present method has been shown to be an accurate, precise analytical method.

The results of measurement on urine samples from mucopolysaccharidosis patients are shown in Tables 14 and 15.

TABLE 14

| | | Concentarations | | | Composition | | |
|---|---|---|---|---|---|---|---|
| Sample | | (μg/mg creatinine) | | | (%) | | Creatinine |
| No. | Data | MSD | DSD | Total | MSD | DSD | (mg/mL) |
| 1 | MPS I | 21 | 5.1 | 26 | 80 | 20 | 0.1324 |
| 2 | MPS I | 14 | 3.5 | 17 | 79 | 21 | 0.244 |
| 3 | MPS I | 5.1 | 1.1 | 6.3 | 82 | 18 | 0.107 |
| 4 | MPS II | 11 | 3.5 | 14 | 75 | 25 | 0.111 |
| 5 | MPS II | 12 | 3.9 | 16 | 76 | 24 | 0.633 |
| 6 | MPS II | 2.3 | 0.91 | 3.2 | 71 | 29 | 0.836 |
| 7 | MPS IIIA | 38 | 10 | 48 | 80 | 20 | 0.0288 |
| 8 | MPS IIIA | 8.7 | 2.8 | 12 | 75 | 25 | 0.172 |
| 9 | MPS IIIA | 22 | 6.5 | 29 | 77 | 23 | 0.054 |
| 10 | MPS IIIB | 14 | 4.7 | 19 | 75 | 25 | 0.188 |
| 11 | MPS IIIB | 7.2 | 2.6 | 9.8 | 74 | 26 | 0.47 |
| 12 | MPS IIIB | 79 | 61 | 140 | 56 | 44 | 0.105 |
| 13 | MPS IIIC | 5.2 | 2.4 | 7.6 | 69 | 31 | 0.463 |
| 14 | MPS IIIC | 2.1 | 1.0 | 3.1 | 67 | 33 | 0.765 |
| 15 | MPS IIIC | 30 | 6.1 | 37 | 83 | 17 | 0.493 |
| 16 | MPS IVA | 19 | 18 | 37 | 50 | 50 | 0.468 |
| 17 | MPS IVA | 4.2 | 3.2 | 7.4 | 57 | 43 | 0.688 |
| 18 | MPS IVA | 13 | 12 | 25 | 51 | 49 | 1.38 |
| 19 | MPS IVB | 37 | 13 | 50 | 74 | 26 | 0.105 |
| 20 | MPS IVB | 6.1 | 2.6 | 8.8 | 70 | 30 | 0.797 |
| 21 | MPS IVB | 15 | 4.8 | 20 | 76 | 24 | 0.2711 |
| 22 | MPS VI | 4.5 | 3.3 | 7.8 | 58 | 42 | 0.799 |
| 23 | MPS VI | 4.3 | 1.9 | 6.2 | 69 | 31 | 0.304 |
| 24 | MPS VI | 3.9 | 2.3 | 6.1 | 63 | 37 | 0.618 |
| 25 | MPS VII | 2.8 | 0.88 | 3.7 | 76 | 24 | 0.193 |
| 26 | MPS VII | 22 | 8.8 | 30 | 71 | 29 | 0.694 |
| 27 | MPS VII | 1.9 | 0.74 | 2.7 | 72 | 28 | 0.43 |
| 28 | Adult control 1 | 0.63 | 0.27 | 0.90 | 70 | 30 | 1.0319 |
| 29 | Adult control 2 | 0.53 | 0.37 | 0.89 | 59 | 41 | 2.2735 |
| 30 | Adult control 3 | 0.46 | 0.19 | 0.65 | 71 | 29 | 1.9874 |
| 31 | Adult control 4 | 0.52 | 0.24 | 0.76 | 69 | 31 | 2.1103 |
| 32 | Adult control 5 | 1.0 | 0.34 | 1.3 | 74 | 26 | 0.7045 |
| 33 | Adult control 6 | 0.28 | 0.15 | 0.43 | 64 | 36 | 3.1815 |
| 34 | Adult control 7 | 0.44 | 0.25 | 0.69 | 64 | 36 | 2.0811 |
| 35 | Adult control 8 | 0.49 | 0.27 | 0.76 | 65 | 35 | 2.0401 |
| 36 | Adult control 9 | 0.49 | 0.22 | 0.71 | 69 | 31 | 1.9045 |
| 37 | Adult control 10 | 0.72 | 0.23 | 1.0 | 76 | 24 | 1.3672 |
| 38 | Adult control 11 | 0.53 | 0.35 | 0.88 | 60 | 40 | 2.6606 |
| 39 | Adult control 12 | 0.47 | 0.30 | 0.77 | 61 | 39 | 1.7903 |

TABLE 15

| | | Concentarations | | | | Composition | | | |
|---|---|---|---|---|---|---|---|---|---|
| Sample | | (ng/mg creatinine) | | | | (%) | | | Creatinine |
| No. | Data | ΔDiHS-0S | ΔDiHS-NS | ΔDi-4S, -6S* | Total | ΔDiHS-0S | ΔDiHS-NS | ΔDi-4S, -6S* | (mg/mL) |
| 1 | MPS I | 110000 | 23000 | 580000 | 713000 | 15 | 3 | 81 | 0.1324 |
| 2 | MPS I | 98000 | 30000 | 980000 | 1108000 | 9 | 3 | 88 | 0.244 |
| 3 | MPS I | 15000 | 3100 | 41000 | 59100 | 25 | 5 | 69 | 0.107 |
| 4 | MPS II | 70000 | 13000 | 200000 | 283000 | 25 | 5 | 71 | 0.111 |
| 5 | MPS II | 63000 | 25000 | 440000 | 528000 | 12 | 5 | 83 | 0.633 |
| 6 | MPS II | 950 | 400 | 2600 | 3950 | 24 | 10 | 66 | 0.836 |
| 7 | MPS IIIA | 330000 | 66000 | 32000 | 428000 | 77 | 15 | 7 | 0.0288 |
| 8 | MPS IIIA | 110000 | 21000 | 18000 | 149000 | 74 | 14 | 12 | 0.172 |
| 9 | MPS IIIA | 240000 | 48000 | 35000 | 323000 | 74 | 15 | 11 | 0.054 |
| 10 | MPS IIIB | 170000 | 53000 | 32000 | 255000 | 67 | 21 | 13 | 0.188 |
| 11 | MPS IIIB | 110000 | 36000 | 26000 | 172000 | 64 | 21 | 15 | 0.47 |
| 12 | MPS IIIB | 260000 | 130000 | 2800000 | 3190000 | 8 | 4 | 88 | 0.105 |
| 13 | MPS IIIC | 63000 | 20000 | 19000 | 102000 | 62 | 20 | 19 | 0.463 |
| 14 | MPS IIIC | 30000 | 8400 | 5500 | 43900 | 68 | 19 | 13 | 0.765 |
| 15 | MPS IIIC | 3200 | 1300 | 5300 | 9800 | 33 | 13 | 54 | 0.493 |
| 16 | MPS IVA | 1400 | 750 | 19000 | 21150 | 7 | 4 | 90 | 0.468 |
| 17 | MPS IVA | 550 | 200 | 2900 | 3650 | 15 | 5 | 79 | 0.688 |
| 18 | MPS IVA | 1600 | 1000 | 15000 | 17600 | 9 | 6 | 85 | 1.38 |
| 19 | MPS IVB | 2200 | 760 | 4200 | 7160 | 31 | 11 | 59 | 0.105 |
| 20 | MPS IVB | 650 | 340 | 1300 | 2290 | 28 | 15 | 57 | 0.797 |
| 21 | MPS IVB | 1800 | 700 | 5500 | 8000 | 23 | 9 | 69 | 0.2711 |
| 22 | MPS VI | 2100 | 1000 | 160000 | 163100 | 1 | 1 | 98 | 0.799 |
| 23 | MPS VI | 1700 | 630 | 110000 | 112330 | 2 | 1 | 98 | 0.304 |
| 24 | MPS VI | 1900 | 940 | 140000 | 142840 | 1 | 1 | 98 | 0.618 |
| 25 | MPS VII | 470 | 140 | 980 | 1590 | 30 | 9 | 62 | 0.193 |
| 26 | MPS VII | 45000 | 20000 | 190000 | 255000 | 18 | 8 | 75 | 0.694 |
| 27 | MPS VII | 7000 | 1800 | 8100 | 16900 | 41 | 11 | 48 | 0.43 |
| 28 | Adult control 1 | 510 | 190 | 610 | 1310 | 39 | 15 | 47 | 1.0319 |
| 29 | Adult control 2 | 700 | 290 | 1100 | 2090 | 33 | 14 | 53 | 2.2735 |
| 30 | Adult control 3 | 440 | 170 | 650 | 1260 | 35 | 13 | 52 | 1.9874 |
| 31 | Adult control 4 | 440 | 150 | 900 | 1490 | 30 | 10 | 60 | 2.1103 |
| 32 | Adult control 5 | 540 | 170 | 1200 | 1910 | 28 | 9 | 63 | 0.7045 |
| 33 | Adult control 6 | 310 | 140 | 690 | 1140 | 27 | 12 | 61 | 3.1815 |
| 34 | Adult control 7 | 430 | 160 | 720 | 1310 | 33 | 12 | 55 | 2.0811 |
| 35 | Adult control 8 | 540 | 230 | 1200 | 1970 | 27 | 12 | 61 | 2.0401 |
| 36 | Adult control 9 | 450 | 170 | 840 | 1460 | 31 | 12 | 58 | 1.9045 |
| 37 | Adult control 10 | 370 | 130 | 700 | 1200 | 31 | 11 | 58 | 1.3672 |
| 38 | Adult control 11 | 750 | 380 | 1900 | 3030 | 25 | 13 | 63 | 2.6606 |
| 39 | Adult control 12 | 530 | 200 | 610 | 1340 | 40 | 15 | 46 | 1.7903 |

*ΔDi-4S, 6S: These disaccharides were produced from DS and HS(6S).

As is clear from Tables 14 and 15, the method of the present invention has been found to be useful in an assay of a clinical sample and also in screening. Mucopolysaccharidosis types I, II, III cases showed high HS concentrations, and a mucopolysaccharidosis type VI case showed a high value of ΔDi-4S,6S concentration.

In particular, KS-derived DSD ratio differs between mucopolysaccharidosis type IV A (No. 16 to 18 in Table 14) and mucopolysaccharidosis type IV B (No. 19 to 21 in Table 14). That is, type IV A showed a high DSD ratio. Therefore, analysis of compositional ratio can distinguish between type IV A and type IV B.

As described above, with the present method, KS, HS, and DS levels can be analyzed simultaneously. If some correlation is identified in future research between age, pathological conditions, etc. of a patient and KS, HS, and DS levels, it is believed that a single assay provides simultaneous diagnosis of different types of mucopolysaccharidoses.

1. A diagnostic method of mucopolysaccharidoses including the following steps (1) and (2):
(1) a step including (a) filtering a biological sample with an ultrafiltration filter, digesting the sample on the filter with a glycosaminoglycan-specific enzyme, centrifuging the digested sample to obtain a filtrate, or (b) digesting a biological sample with a glycosaminoglycan-specific enzyme, filtering the sample with an ultrafiltration filter, applying the filtrate obtained by (a) or (b) to a liquid chromatograph/mass spectrometer, and analyzing glycosaminoglycan-derived disaccharides, and
(2) a step of diagnosing a subject as having mucopolysaccharidosis, chemically diagnosing effect of treatment of mucopolysaccharidoses, or determining types of mucopolysaccharidoses, on the basis of quantitative concentration data and disaccharide composition obtained by step (1).

2. The method according to claim 1, wherein, in step (1), liquid chromatography is performed under such conditions that the analytical column is a carbon graphite column and an alkaline solution is employed as a mobile phase, to thereby elute glycosaminoglycan-derived disaccharides at optimal elution positions that facilitate the MS analysis.

3. The method according to claim 1, wherein, in step (1), the disaccharides are produced through use of a solution containing, as the glycosaminoglycan-specific degrading enzyme, keratanase II, heparitinase, and chondroitinase B; and keratan sulfate, heparan sulfate, dermatan sulfate are analyzed simultaneously.

4. The method according to claim 1, wherein, in step (1), the disaccharides are produced through use of a solution containing, as the glycosaminoglycan-specific degrading enzyme, keratanase II, heparitinase, and chondroitinase B; and keratan sulfate, heparan sulfate, dermatan sulfate are analyzed simultaneously.

5. The method according to claim 1, wherein, in step (1), the biological sample is selected from among plasma, serum, blood, urine, and body fluid.
